(19) United States
(12) Patent Application Publication
(10) Pub. No.: US 2024/0206753 A1
(43) Pub. Date: Jun. 27, 2024

(54) BIOLOGICAL INFORMATION MEASUREMENT DEVICE, BIOLOGICAL INFORMATION MEASUREMENT METHOD, AND BIOLOGICAL INFORMATION MEASUREMENT SYSTEM

(71) Applicant: SEIKO EPSON CORPORATION, TOKYO (JP)

(72) Inventors: Ayae SAWADO, Kai-shi (JP); Hiroki KOBAYASHI, HARA-MURA (JP)

(21) Appl. No.: 18/391,765

(22) Filed: Dec. 21, 2023

(30) Foreign Application Priority Data
Dec. 22, 2022 (JP) 2022-205230

(51) Int. Cl. A61B 5/024; A61B 5/00; A61B 5/1455

(52) U.S. Cl. CPC A61B 5/02433; A61B 5/14552; A61B 5/4818; A61B 2560/0252

(57) ABSTRACT

A biological information measurement device includes: a light emitting unit including a light emitting element that emits light; a light receiving unit configured to receive the light passing through a living body and generate a detection signal; a temperature sensor configured to measure at least one of an environmental temperature and a body temperature of the living body and generate temperature data; and a controller configured to adjust a light emitting intensity of the light emitting element. The controller calculates a temperature change rate using the temperature data, and adjusts the light emitting intensity based on the temperature change rate.

The present application is based on, and claims priority from JP Application Serial Number 2022-205230, filed Dec. 22, 2022, the disclosure of which is hereby incorporated by reference herein in its entirety.

### BACKGROUND

### 1. Technical Field

The present disclosure relates to a biological information measurement device, a biological information measurement method, and a biological information measurement system.

### 2. Related Art

A measurement device that non-invasively measures biological information of a subject is known. A measurement device described in JP-A-2022-86227 measures a pulse wave and an oxygen saturation concentration. The measurement device emits light from a plurality of light emitting units to a living body. The measurement device receives light emitted from the living body and acquires a detection signal.

A signal intensity of the detection signal fluctuates depending on a change in an environmental temperature or a body temperature. When the signal intensity fluctuates, measurement accuracy of the measurement device decreases.

### SUMMARY

A biological information measurement device of the present disclosure includes: a light emitting unit including a light emitting element that emits light; a light receiving unit configured to receive the light passing through a living body and generate a detection signal; a temperature sensor configured to measure at least one of an environmental temperature and a body temperature of the living body and generate temperature data; and a controller configured to adjust a light emitting intensity of the light emitting element. The controller calculates a temperature change rate using the temperature data, and adjusts the light emitting intensity based on the temperature change rate.

A biological information measurement method of the present disclosure is a biological information measurement method of measuring biological information of a living body. The biological information measurement method includes: measuring at least one of an environmental temperature and a body temperature of the living body and generating temperature data; emitting light to the living body; receiving the light passing through the living body and generating a detection signal; calculating a temperature change rate using the temperature data; and adjusting, based on the temperature change rate, a light emitting intensity when the light is emitted.

A biological information measurement system of the present disclosure includes: a biological information measurement device including a light emitting unit including a light emitting element that emits light, a light receiving unit configured to receive the light passing through a living body and generate a detection signal, a temperature sensor configured to measure at least one of an environmental temperature and a body temperature of the living body and generate temperature data, a controller configured to calculate a temperature change rate using the temperature data, and adjust a light emitting intensity of the light emitting element based on the calculated temperature change rate, and a communication unit configured to transmit the detection signal; and a control device including a terminal communication circuit configured to receive the detection signal, and an analysis controller configured to analyze biological information of the living body using the detection signal.

### BRIEF DESCRIPTION OF THE DRAWINGS

FIG. 1 is a diagram showing a schematic configuration of a measurement system.

FIG. 2 is a diagram showing a schematic configuration of a measurement surface.

FIG. 3 is a diagram showing a block configuration of a measurement device.

FIG. 4 is a diagram showing an example of light emission of a light emitting pattern of a light emitting element.

FIG. 5 is a diagram schematically showing a detection signal.

FIG. 6 is a diagram showing a relationship between a frequency and a signal intensity of each detection signal in a predetermined time.

FIG. 7 is a diagram showing a measurement result of a pulse wave intensity.

FIG. 8 is a flowchart for measuring a pulse wave.

FIG. 9 is a flowchart for adjusting a light emitting intensity.

FIG. 10 is a flowchart for adjusting the light emitting intensity.

FIG. 11 is a diagram showing the schematic configuration of the measurement surface.

FIG. 12 is a diagram showing the block configuration of the measurement device.

FIG. 13 is a flowchart for measuring the pulse wave.

FIG. 14 is a flowchart for adjusting the light emitting intensity.

FIG. 15 is a diagram showing a block configuration of the measurement system.

FIG. 16 is a flowchart for measuring the detection signal.

FIG. 17 is a diagram showing an operation of a tablet terminal in the measurement system.

### DESCRIPTION OF EMBODIMENTS

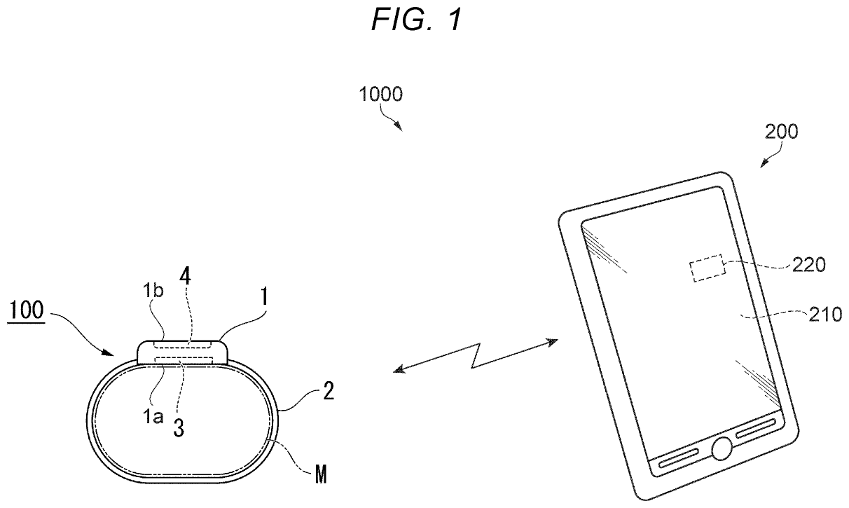

FIG. 1 shows a schematic configuration of a measurement system 1000. The measurement system 1000 evaluates an irregular pulse. The measurement system 1000 evaluates the irregular pulse using a pulse wave as an index. The irregular pulse is evaluated using a beat interval or the like. When the irregular pulse is evaluated using the measurement system 1000, the pulse wave is measured for 7 days or more. The measurement system 1000 may evaluate sleep apnea syndrome. The sleep apnea syndrome is represented as SAS. The measurement system 1000 evaluates the sleep apnea syndrome using an oxygen saturation concentration (SpO₂) in blood as an index. The oxygen saturation concentration in blood is represented as an oxygen saturation concentration. When the evaluation of the sleep apnea syndrome is performed using the measurement system 1000, the oxygen saturation concentration is measured over one to three nights. The sleep apnea syndrome is evaluated from a low-concentration period indicating an oxygen saturation concentration lower than a predetermined value or the number of times of the low-concentration period. The measurement system 1000 corresponds to an example of a biological information measurement system.

The measurement system 1000 includes a measurement device 100 and a tablet terminal 200. The measurement device 100 and the tablet terminal 200 are communicably connected to each other. The measurement system 1000 shown in FIG. 1 communicably connects the measurement device 100 and the tablet terminal 200 in a wireless manner. The connection between the measurement device 100 and the tablet terminal 200 is not limited to wireless connection. The measurement device 100 and the tablet terminal 200 may be connected in a wired manner. The measurement device 100 corresponds to an example of a biological information measurement device. The tablet terminal 200 corresponds to an example of a control device.

The measurement device 100 measures biological information of a user M such as a human or various types of data related to the biological information. The user M corresponds to an example of a living body. The measurement device 100 is a watch type portable device worn on a measurement site of the user M. The measurement device 100 shown in FIG. 1 is worn on, for example, the wrist of the user M. The measurement device 100 measures biological information such as pulse waves including pulse wave intervals, oxygen saturation concentrations, or the like over time. The pulse wave interval is represented as a post pacing interval (PPI). The pulse wave indicates a change with time in a volume of a blood vessel in conjunction with the heart beat. The oxygen saturation concentration indicates a proportion of hemoglobin bound to oxygen to hemoglobin in arterial blood of the user M. The oxygen saturation concentration is an index for evaluating a breathing function of the user M. The measurement device 100 may measure the biological information other than the pulse wave and the oxygen saturation concentration. The measurement device 100 measures, for example, a glucose concentration in the arterial blood and an alcohol concentration in the arterial blood. The measurement device 100 includes a housing 1 and a belt 2. The housing 1 accommodates a detection unit 3 and a display panel 4.

The housing 1 is an exterior housing accommodating a unit or the like provided in the measurement device 100. The housing 1 has a measurement surface 1a and a display surface 1b. The measurement surface 1a is a surface facing the measurement site of the user M. The measurement surface 1a is in contact with the measurement site of the user M. The display surface 1b is a surface visible to the user M. In addition to the detection unit 3 and the display panel 4, the housing 1 accommodates a control unit 30, a memory 40, or the like, which will be described later.

The belt 2 is a member that is used when the user M wears the housing 1 on the measurement site. The belt 2 is attached to, for example, a side surface of the housing 1. The belt 2 is wound around the measurement site, and thereby the housing 1 is worn on the measurement site of the user M. The measurement device 100 shown in FIG. 1 includes the belt 2, but is not limited thereto. The measurement device 100 may not include the belt 2. The measurement device 100 may be attached to a chest, an arm, or the like of the user M using a tape or the like.

The detection unit 3 is disposed on the measurement surface 1a of the housing 1. The detection unit 3 is disposed at a position facing the measurement site of the user M. The detection unit 3 acquires various types of data used when measuring the biological information.

The display panel 4 is disposed on the display surface 1b of the housing 1. The display panel 4 is visible to the user M. The display panel 4 displays various types of the measured biological information. The display panel 4 may display information such as a reliability index of the biological information and time other than the biological information. The display panel 4 corresponds to an example of a display unit.

The tablet terminal 200 evaluates the irregular pulse. The tablet terminal 200 evaluates the sleep apnea syndrome. The tablet terminal 200 acquires various types of data transmitted from the measurement device 100. The tablet terminal 200 evaluates the irregular pulse using the various types of data. The tablet terminal 200 evaluates the sleep apnea syndrome using the various types of data. The measurement system 1000 shown in FIG. 1 includes the tablet terminal 200, but is not limited thereto. The measurement system 1000 may include a personal computer, a smartphone, a dedicated terminal for biological information analysis, or the like, instead of the tablet terminal 200. The tablet terminal 200 includes a display 210 and a terminal control unit 220.

The display 210 displays an evaluation result of the irregular pulse. The display 210 may display an evaluation result of the sleep apnea syndrome. The display 210 may display the biological information such as an oxygen saturation concentration and a pulse interval in a chart.

The terminal control unit 220 evaluates the irregular pulse. The terminal control unit 220 evaluates the sleep apnea syndrome. The terminal control unit 220 acquires the various types of data transmitted from the measurement device 100. The terminal control unit 220 evaluates the irregular pulse using the various types of data. The terminal control unit 220 evaluates the sleep apnea syndrome using the various types of data. The terminal control unit 220 generates chart data related to the biological information such as a pulse wave and an oxygen saturation concentration. The terminal control unit 220 controls the display 210, and causes the display 210 to display evaluation results of various types of biological information and the biological information in a chart. The terminal control unit 220 corresponds to an example of an analysis controller.

### First Embodiment

A first embodiment shows a configuration of a first measurement device 100a which is an example of the measurement device 100. The first embodiment shows a biological information measurement method using the first measurement device 100a. The first measurement device 100a measures a pulse wave and an oxygen saturation concentration. The first measurement device 100a transmits the measured pulse wave and oxygen saturation concentration to the tablet terminal 200. The tablet terminal 200 receives the pulse wave and the oxygen saturation concentration, and evaluates an irregular pulse, sleep apnea syndrome, or the like.

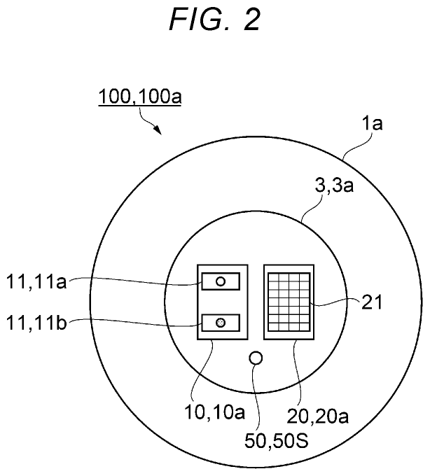

FIG. 2 shows a schematic configuration of the measurement surface 1a of the measurement device 100. FIG. 2 shows a schematic configuration when the measurement surface 1a of the first measurement device 100a, which is an example of the measurement device 100, is viewed from the outside. The measurement surface 1a shown in FIG. 2 is formed in a circular shape, but is not limited thereto. The measurement surface 1a may be formed in various shapes such as a square shape and an elliptical shape. A first detection unit 3a is disposed on the measurement surface 1a. The first detection unit 3a is an example of the detection unit 3. The first detection unit 3a includes a first light emitting element unit 10a and a first light receiving element unit 20a. The first light emitting element unit 10a is an example of a light emitting element unit 10. The first light receiving element unit 20a is an example of a light receiving element unit 20. The light emitting element unit 10 corresponds to an example of a light emitting unit. The light receiving element unit 20 corresponds to an example of a light receiving unit. The first detection unit 3a includes a detector 50S of a temperature detection sensor 50. The temperature detection sensor 50 will be described later.

The first light emitting element unit 10a emits light toward the measurement site of the user M. The first light emitting element unit 10a shown in FIG. 2 includes two light emitting elements 11. The two light emitting elements 11 are a red light emitting element 11a and an infrared light emitting element 11b. The red light emitting element 11a and the infrared light emitting element 11b emit light in different wavelength ranges. An arrangement of the two light emitting elements 11 is appropriately set. The number of the light emitting elements 11 is not limited to two. Three or more light emitting elements 11 may be provided on the first light emitting element unit 10a.

The light emitting element 11 is implemented by a bare chip type or a shell type light emitting diode (LED). The light emitting element 11 may be a laser diode. A configuration of the light emitting element 11 is appropriately set according to a wavelength range of light to be emitted.

The first light receiving element unit 20a receives various types of light emitted by the first light emitting element unit 10a. The first light receiving element unit 20a includes a light receiving element 21 that receives various types of light passing through the user M. The light receiving element 21 receives transmitted light or reflected light of the light emitted by the first light emitting element unit 10a. The transmitted light is light transmitted through the user M. The reflected light is light reflected in the user M and transmitted through the inside of the user M. The transmitted light and the reflected light pass through the inside of the user M. The light receiving element 21 includes one or a plurality of photodiodes.

The detector 50S of the temperature detection sensor 50 is in contact with the skin of the user M. The detector 50S is a measurement unit of the temperature detection sensor 50. When the detector 50S is provided at a position in contact with the user M, the temperature detection sensor 50 detects a body temperature of the user M. The position of the detector 50S is not limited to the position shown in FIG. 2. When the temperature detection sensor 50 measures an outside air temperature, the detector 50S is provided at a position not facing the user M.

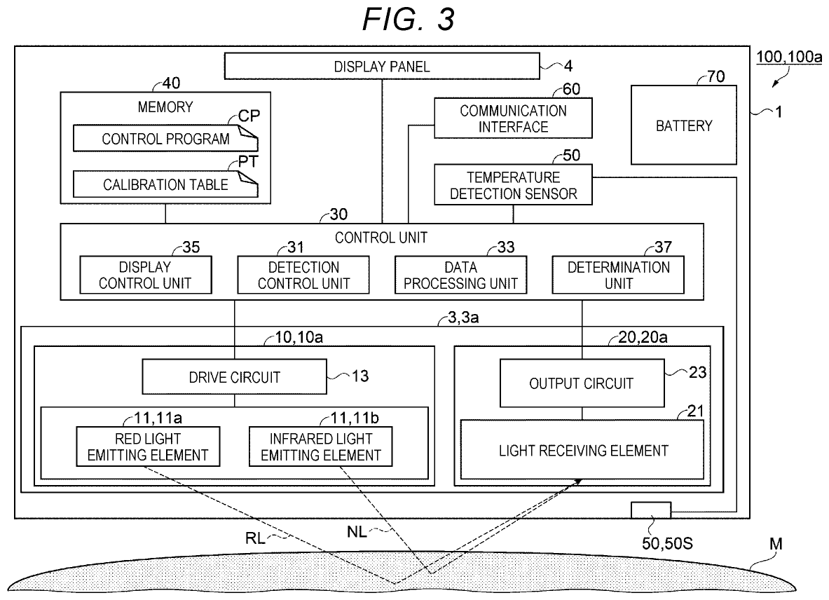

FIG. 3 shows a block configuration of the measurement device 100. FIG. 3 shows a block configuration of the first measurement device 100a which is an example of the measurement device 100. FIG. 3 shows the first measurement device 100a excluding the belt 2. The first measurement device 100a accommodates various units or the like in the housing 1. The first measurement device 100a includes the first detection unit 3a, the control unit 30, the memory 40, the display panel 4, the temperature detection sensor 50, a communication interface 60, and a battery 70.

The first detection unit 3a is an optical sensor module that detects, as a detection signal, data related to biological information measured using light in various wavelength ranges. The first detection unit 3a includes the first light emitting element unit 10a and the first light receiving element unit 20a.

The first light emitting element unit 10a includes two light emitting elements 11 and a drive circuit 13. The two light emitting elements 11 shown in FIG. 3 are the red light emitting element 11a and the infrared light emitting element 11b.

The red light emitting element 11a emits red light RL toward the measurement site of the user M. The red light emitting element 11a emits the red light RL in a wavelength range from 600 nm to 800 nm toward the measurement site. The red light RL is, for example, light having a peak wavelength of 660 nm.

The infrared light emitting element 11b emits infrared light NL toward the measurement site of the user M. The infrared light emitting element 11b emits the infrared light NL in a wavelength range from 800 nm to 1300 nm toward the measurement site. The infrared light NL is, for example, near infrared light having a peak wavelength of 905 nm.

The drive circuit 13 drives a plurality of light emitting elements 11. The drive circuit 13 causes the plurality of light emitting elements 11 to emit light under the control of the control unit 30. The drive circuit 13 shown in FIG. 3 causes the red light emitting element 11a and the infrared light emitting element 11b to emit light.

The first light receiving element unit 20a includes the light receiving element 21 and an output circuit 23. The light receiving element 21 receives reflected light emitted by the light emitting element 11 and reflected by the measurement site of the user M. The light receiving element 21 receives the red light RL and the infrared light NL reflected by the measurement site of the user M. The light receiving element 21 alternately receives the red light RL and the infrared light NL in a time-division manner. The light receiving element 21 may be divided into two regions. Each of the two regions receives either the red light RL or the infrared light NL. The light receiving element 21 may be divided into a plurality of regions using an optical filter (not shown). The light receiving element 21 receives at least one of the red light RL and the infrared light NL through the optical filter.

The first light receiving element unit 20a shown in FIG. 3 receives reflected light of the red light RL and reflected light of the infrared light NL, but is not limited thereto. The first light receiving element unit 20a may receive the red light RL transmitted through the user M and the infrared light NL transmitted through the user M. The first light receiving element unit 20a receives the transmitted light of the red light RL and the transmitted light of the infrared light NL.

The output circuit 23 generates a detection signal based on the light received by the light receiving element 21. The output circuit 23 outputs the detection signal to the control unit 30. The output circuit 23 generates the detection signal by performing processing such as analog-to-digital conversion on light receiving intensity data of the light received by the light receiving element 21. The output circuit 23 generates a red light detection signal based on the red light RL received by the light receiving element 21. The output circuit 23 generates an infrared light detection signal based on the infrared light NL received by the light receiving element 21. The red light detection signal and the infrared light detection signal are examples of detection signals.

The control unit 30 is a controller that controls operations of various units. The control unit 30 is, for example, a processor including a central processing unit (CPU). The control unit 30 may include one or a plurality of processors. The control unit 30 may include a semi-conductor memory such as a random access memory (RAM) or a read only memory (ROM). The semi-conductor memory functions as a work area of the control unit 30. The control unit 30 functions as a detection control unit 31, a data processing unit 33, and a display control unit 35, and a determination unit 37 by executing a control program CP stored in the memory 40. The detection control unit 31, the data processing unit 33, the display control unit 35, and the determination unit 37 are functional units. The control unit 30 controls the first light emitting element unit 10a and the first light receiving element unit 20a by the functional units. The control unit 30 corresponds to an example of a controller.

The detection control unit 31 controls the first light emitting element unit 10a and the first light receiving element unit 20a. The detection control unit 31 adjusts light emitting intensity of the light emitting element 11 via the drive circuit 13. The detection control unit 31 adjusts the light emitting intensity by adjusting the number of times of light emission, a light emitting pattern, and a light emitting intensity per pulse. The detection control unit 31 controls a light receiving timing, a light receiving time, digital-to-analog conversion, or the like of various types of light for the first light receiving element unit 20a. The detection control unit 31 controls the first light emitting element unit 10a and the first light receiving element unit 20a to cause the first detection unit 3a to generate the red light detection signal and the infrared light detection signal. The detection control unit 31 controls the light emitting element 11 based on a determination result of the determination unit 37. The control of the detection control unit 31 based on the determination result of the determination unit 37 will be described later.

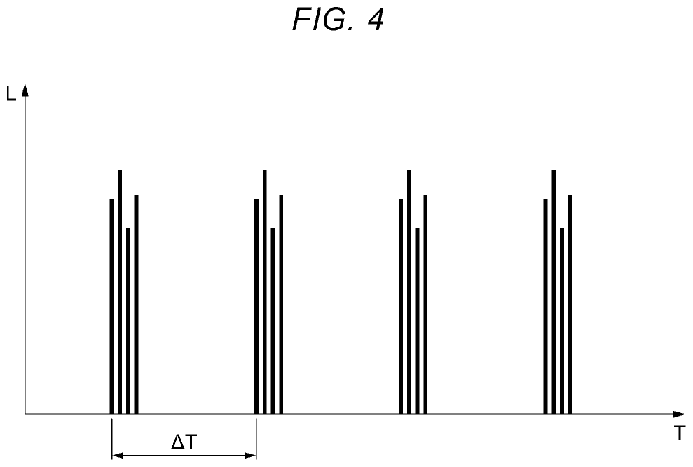

FIG. 4 shows an example of light emission of a light emitting pattern of the light emitting element 11. A horizontal axis in FIG. 4 indicates time T. A vertical axis in FIG. 4 indicates a light amount L. The detection control unit 31 causes the light emitting element 11 to emit light at a predetermined light emitting period ΔT. The detection control unit 31 causes the light emitting element 11 to emit a plurality of pulses for each light emitting period ΔT. In the case of FIG. 4, the light emitting element 11 emits four pulses for each light emitting period ΔT. The number of pulses is not limited to four. A light emission amount per pulse is appropriately set. The light emitting intensity for each light emitting period ΔT is a sum of the light emission amounts of the plurality of pulses. The light emitting intensity of the light emitting element 11 is determined based on the number of pulses, the light emission amount of one pulse, and a pulse interval. The light emitting pattern of each light emitting element 11 is appropriately adjusted. For example, the light emitting pattern of the red light emitting element 11a may be the same as or different from the light emitting pattern of the infrared light emitting element 11b.

The data processing unit 33 shown in FIG. 3 processes the detection signal transmitted from the first light receiving element unit 20a. The data processing unit 33 acquires the red light detection signal and the infrared light detection signal from the first light receiving element unit 20a.

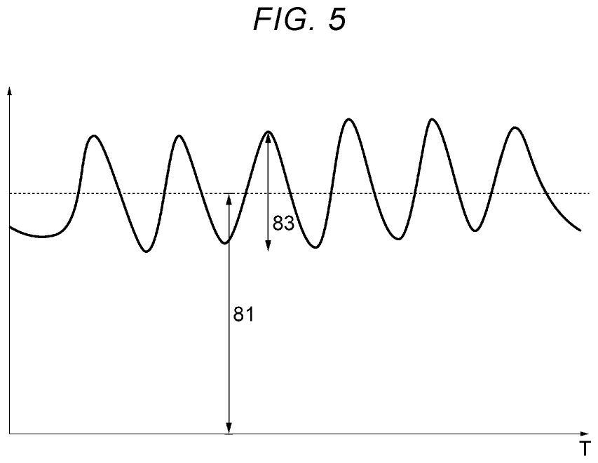

The data processing unit 33 measures a DC component and an AC component from the detection signal. FIG. 5 schematically shows a detection signal. The horizontal axis in FIG. 5 indicates time T. The vertical axis in FIG. 5 indicates a signal intensity of the detection signal. FIG. 5 schematically shows an example of the detection signal output from the output circuit 23.

The detection signal is data of a signal intensity detected at a predetermined measurement time interval. The signal intensity is detected n times per second. n is an integer of 1 or more. For example, n is 16. The signal intensity includes DC component data 81 as a DC component and AC component data 83 as an AC component. The data processing unit 33 separates the DC component data 81 and the AC component data 83 from the signal intensity. The data processing unit 33 measures the AC component data 83 by performing time-frequency analysis.

The data processing unit 33 performs the time-frequency analysis such as short time Fourier transform on the detection signal. The data processing unit 33 analyzes frequency information by performing the short time Fourier transform on the detection signal. The data processing unit 33 obtains a spectrogram in a predetermined frequency range by performing the short time Fourier transform on the detection signal. The predetermined frequency range is a range including the frequency of the pulse wave. The predetermined frequency range is, for example, a range from 0.5 Hz to 2 Hz. The predetermined frequency range is appropriately adjusted according to a wavelength range of light subjected to the short time Fourier transform. The data processing unit 33 performs the short time Fourier transform on the red light detection signal to obtain a red light spectrogram. The data processing unit 33 performs the short time Fourier transform on the infrared light detection signal to obtain an infrared light spectrogram. The data processing unit 33 corresponds to an example of the controller.

The time-frequency analysis executed by the data processing unit 33 is not limited to the short time Fourier transform. The method is not limited as long as the frequency information for the detection signal can be analyzed. The data processing unit 33 may perform, for example, wavelet conversion.

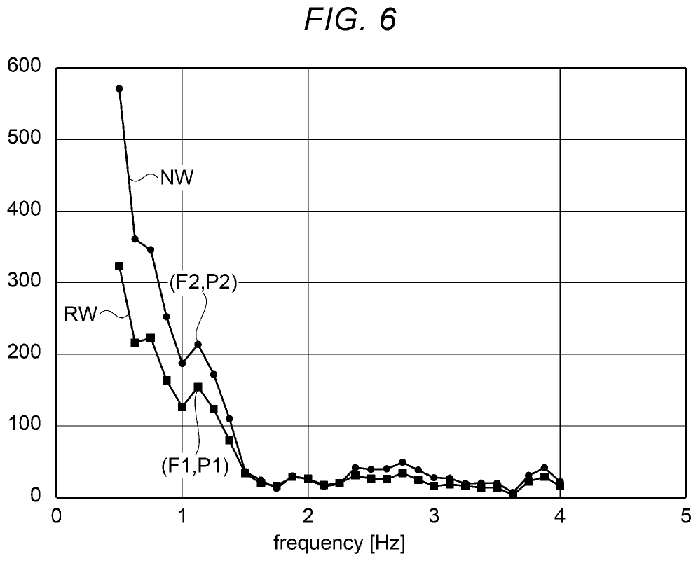

FIG. 6 shows a relationship between a frequency and a signal intensity of each detection signal in a predetermined time. FIG. 6 shows a part of a result of the short time Fourier transform. FIG. 6 shows red light data RW and infrared light data NW in the predetermined time. The red light data RW shown in FIG. 6 indicates a relationship between a frequency and a signal intensity of the red light RL at the predetermined time. The infrared light data NW shown in FIG. 6 indicates a relationship between a frequency and a signal intensity of the infrared light NL at the predetermined time.

The red light data RW indicates a first peak value P1 at a first frequency F1. The first frequency F1 corresponds to a frequency of a pulse wave. The data processing unit 33 acquires the signal intensity at each time at the first frequency F1 as a red light detection signal intensity.

The infrared light data NW indicates a second peak value P2 at a second frequency F2. The second frequency F2 is equal to or approximate to the first frequency F1. The second frequency F2 corresponds to a frequency of a pulse wave. The data processing unit 33 acquires the signal intensity at each time at the second frequency F2 as an infrared light detection signal intensity.

The data processing unit 33 acquires the red light detection signal intensity and the infrared light detection signal intensity at each time. The data processing unit 33 measures a fluctuation component amplitude ratio using the red light detection signal intensity and the infrared light detection signal intensity. The fluctuation component amplitude ratio is a ratio between a red light transmitted amount and an infrared light transmitted amount. The red light transmitted amount is a light amount of the red light RL, which is emitted from the red light emitting element 11a, is transmitted through the measurement site of the user M, and reaches the light receiving element 21. The infrared light transmitted amount is a light amount of the infrared light NL, which is emitted from the infrared light emitting element 11b, is transmitted through the measurement site of the user M, and reaches the light receiving element 21. The fluctuation component amplitude ratio is calculated by the following formula (1).

$$R = ( {AC}_{Red} / {DC}_{Red} ) / ( {AC}_{IR} / {DC}_{IR} ) \qquad ( 1 )$$

Here, R indicates the fluctuation component amplitude ratio. AC_{Red} indicates an intensity of an AC component of the red light detection signal. DC_{Red} indicates an intensity of a DC component of the red light detection signal. AC_{IR} indicates an intensity of an AC component of the infrared light detection signal. DC_{IR} indicates an intensity of a DC component of the infrared light detection signal.

The intensity of the AC component of the red light detection signal is the red light detection signal intensity. The intensity of the DC component of the red light detection signal is a DC component separated from the red light detection signal at the predetermined time. The intensity of the AC component of the infrared light detection signal is the infrared light detection signal intensity. The intensity of the DC component of the infrared light detection signal is a DC component separated from the infrared light detection signal at the predetermined time.

The data processing unit 33 calculates the oxygen saturation concentration based on the calculated fluctuation component amplitude ratio. The data processing unit 33 refers to a calibration table PT stored in the memory 40 to obtain a value of the oxygen saturation concentration corresponding to the fluctuation component amplitude ratio. The data processing unit 33 determines, as the oxygen saturation concentration, the value of the oxygen saturation concentration corresponding to the fluctuation component amplitude ratio. The data processing unit 33 measures the oxygen saturation concentration using the red light detection signal and the infrared light detection signal. The data processing unit 33 measures the oxygen saturation concentration over time. A plurality of oxygen saturation concentrations measured over time are represented as an oxygen saturation concentration data group.

The data processing unit 33 measures a pulse wave using the red light detection signal or the infrared light detection signal.

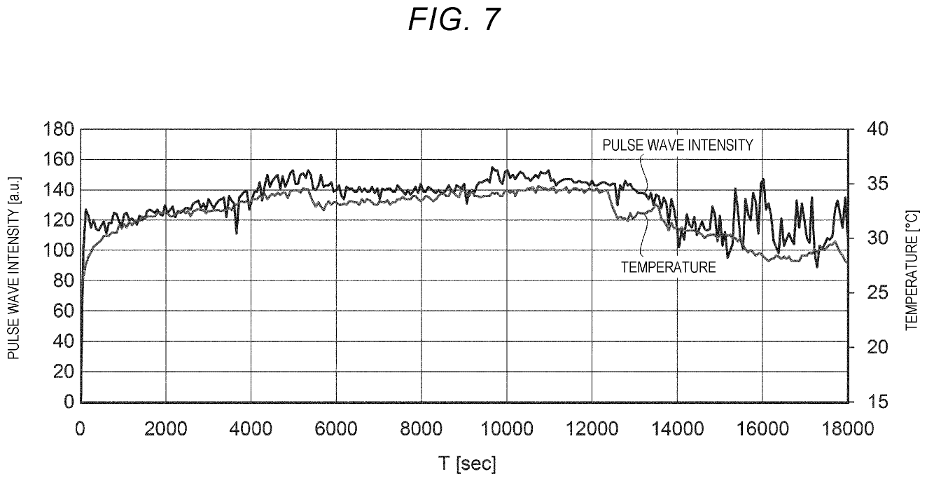

FIG. 7 shows a measurement result of a pulse wave intensity. FIG. 7 shows a change with time of the pulse wave intensity. FIG. 7 shows a change in a body surface temperature of the user M together with the change with time of the pulse wave intensity. The body surface temperature is an example of a body temperature. FIG. 7 shows the pulse wave intensity measured using the infrared light detection signal as an example. The pulse wave intensity measured using the red light detection signal changes similarly to the pulse wave intensity shown in FIG. 7. FIG. 7 shows the pulse wave intensity when the light emitting element 11 emits light with the same light emitting intensity.

As shown in FIG. 7, when the body surface temperature of the user M is stable, variation in the pulse wave intensity is small. A variation in the infrared light detection signal is small. On the other hand, when the body surface temperature of the user M is lowered, the variation in the pulse wave intensity is increased as compared with that when the body surface temperature is stable. Due to the temperature change, disturbance occurs, and the variation in the pulse wave intensity increases. When a temperature change rate increases, the infrared light detection signal varies and the measurement accuracy of the pulse wave decreases. FIG. 7 shows a relationship between the variation in the pulse wave intensity and the body surface temperature, which is an example of the body temperature of the user M. The variation in the pulse wave with respect to a change rate of the outside air temperature occurs similarly to the variation in the pulse wave with respect to the body surface temperature. FIG. 7 shows a case in which the body surface temperature of the user M is lowered, and when the body surface temperature of the user M increases, the variation in the pulse wave intensity also increases.

The data processing unit 33 may calculate a correlation coefficient as an index indicating variation in the red light detection signal and the infrared light detection signal. The correlation coefficient is an index indicating reliability of the red light detection signal and the infrared light detection signal. The correlation coefficient is calculated using the red light detection signal and the infrared light detection signal. The correlation coefficient calculated by the data processing unit 33 is used by the determination unit 37.

The data processing unit 33 calculates a correlation coefficient using the red light detection signal and the infrared light detection signal. The data processing unit 33 calculates a correlation coefficient between red light detection signal data and infrared light detection signal data. The red light detection signal data is a signal intensity data group of the first frequency F1. The infrared light detection signal data is a signal intensity data group of the second frequency F2. The correlation coefficient decreases due to the influence of noise caused by a temperature change. When the temperature change rate is small, the influence of noise caused by disturbance is reduced, and the correlation coefficient is increased. The data processing unit 33 calculates the correlation coefficient using a correlation coefficient calculation formula represented by the following formula (2). The correlation coefficient corresponds to an example of correlation data.

$$r = \frac{{\sum }_{n = 1}^{N} ( {x}_{n} - {x}_{ave} ) ( {y}_{n} - {y}_{a v e} )}{\sqrt{{\sum }_{n = 1}^{N} {( {x}_{n} - {x}_{a v e} )}^{2} {\sum }_{n = 1}^{N} {( {y}_{n} - {y}_{ave} )}^{2}}} \qquad ( 2 )$$

Here, r indicates a correlation coefficient. N indicates the number of red light detection signals used for calculating the correlation coefficient. xₙ indicates a red light detection signal at each measurement time. n is an integer of 1 or more. xₐᵥₑ indicates an average value of the red light detection signals within a predetermined time. yₙ indicates an infrared light detection signal at each measurement time. yₐᵥₑ indicates an average value of the infrared light detection signals within the predetermined time. The predetermined time is, for example, 8 seconds. When the red light detection signal and the infrared light detection signal are measured k times per second, N is 8×k. k is an integer of 1 or more. When the data processing unit 33 calculates the correlation coefficient, one of the red light emitting element 11a and the infrared light emitting element 11b corresponds to an example of a second light emitting element that emits second light. One of the red light detection signal and the infrared light detection signal corresponds to an example of a second detection signal.

The data processing unit 33 outputs the pulse wave and the oxygen saturation concentration to the display control unit 35. The data processing unit 33 outputs the pulse wave and the oxygen saturation concentration to the tablet terminal 200 via the communication interface 60.

The display control unit 35 controls display of the display panel 4. The display control unit 35 causes the display panel 4 to display various images by transmitting display data to the display panel 4. The display control unit 35 acquires the pulse wave and the oxygen saturation concentration from the data processing unit 33 at a predetermined timing. The display control unit 35 generates display data including the pulse wave. The display control unit 35 may generate display data including the oxygen saturation concentration. The display control unit 35 outputs the display data including the pulse wave, the oxygen saturation concentration, or the like to the display panel 4. The display control unit 35 causes the display panel 4 to display the pulse wave and the oxygen saturation concentration based on the display data. The display control unit 35 corresponds to an example of the controller.

The determination unit 37 determines whether the temperature change rate is equal to or greater than a predetermined value. The determination unit 37 acquires temperature data from the temperature detection sensor 50. The determination unit 37 calculates a temperature change amount per unit time as the temperature change rate. The temperature change rate is a negative value when the temperature decreases per unit time. The temperature change rate is a positive value when the temperature increases per unit time. The determination unit 37 compares the temperature change rate with a predetermined change rate threshold. The change rate threshold is an example of the predetermined value. The determination unit 37 determines whether a predetermined temperature change has occurred by comparing the temperature change rate with the change rate threshold. When the determination unit 37 determines that the predetermined temperature change has occurred, the determination unit 37 causes the detection control unit 31 to adjust the light emitting intensity of the light emitting element 11. The determination unit 37 outputs the determination result to the detection control unit 31 to adjust the light emitting intensity of the light emitting element 11. The determination unit 37 may transmit the determination result to the data processing unit 33 or the like. The determination unit 37 corresponds to an example of the controller.

The determination unit 37 compares the temperature change rate with the change rate threshold. The change rate threshold is stored in advance in the memory 40. The determination unit 37 reads the change rate threshold from the memory 40. The determination unit 37 may compare a plurality of change rate thresholds with the temperature change rate. For example, when the temperature change rate is a negative value, the determination unit 37 compares a first change rate threshold with the temperature change rate. When the temperature change rate is a positive value, the determination unit 37 compares a second change rate threshold with the temperature change rate. The determination unit 37 may compare one change rate threshold with the temperature change rate. For example, the determination unit 37 compares the change rate threshold with an absolute value of the temperature change rate.

The determination unit 37 may calculate a detection signal ratio of the detection signal. The detection signal ratio is a ratio of the AC component to the DC component of the detection signal. The determination unit 37 adjusts the light emitting intensity of the light emitting element 11 based on the temperature change rate and the detection signal ratio. For example, when the absolute value of the temperature change rate is equal to or greater than the change rate threshold, the determination unit 37 calculates a detection signal ratio. The determination unit 37 compares the detection signal ratio with a predetermined ratio threshold. When the detection signal ratio is smaller than the ratio threshold, the determination unit 37 causes the detection control unit 31 to adjust the light emitting intensity of the light emitting element 11. The detection signal ratio corresponds to an example of a signal ratio. The ratio threshold corresponds to an example of a predetermined value.

The determination unit 37 may acquire the correlation coefficient calculated by the data processing unit 33. The determination unit 37 adjusts the light emitting intensity of the light emitting element 11 based on the temperature change rate and the correlation coefficient. For example, when the absolute value of the temperature change rate is equal to or greater than the change rate threshold, the determination unit 37 compares a correlation coefficient threshold set in advance with the correlation coefficient. When the correlation coefficient is smaller than the correlation coefficient threshold, the determination unit 37 causes the detection control unit 31 to adjust the light emitting intensity of the light emitting element 11. When the correlation coefficient is equal to or greater than the correlation coefficient threshold, the determination unit 37 may not cause the detection control unit 31 to adjust the light emitting intensity of the light emitting element 11.

The detection control unit 31 adjusts the light emitting intensity of the light emitting element 11 based on the temperature change rate which is the determination result of the determination unit 37. The detection control unit 31 increases or decreases the light emitting intensity of the light emitting element 11. The detection control unit 31 increases the light emitting intensity of the light emitting element 11 by increasing at least one of the number of pulses during light emission and the light emission amount per pulse. The detection control unit 31 decreases the light emitting intensity of the light emitting element 11 by decreasing at least one of the number of pulses during light emission and the light emission amount per pulse.

When the temperature change rate is a negative value, the detection control unit 31 increases the light emitting intensity of the light emitting element 11. When the temperature change rate is a negative value and is equal to or smaller than the first change rate threshold, the detection control unit 31 increases the light emitting intensity of the light emitting element 11. As the light emitting intensity of the light emitting element 11 increases, the intensity of the AC component of the detection signal increases, and the variation in the pulse wave decreases. Since the variation in the pulse wave decreases, a decrease in the measurement accuracy of the pulse wave is prevented. In addition, a decrease in the measurement accuracy of the oxygen saturation concentration is prevented.

When the temperature change rate is a positive value, the detection control unit 31 decreases the light emitting intensity of the light emitting element 11. When the temperature change rate is a positive value and is equal to or greater than the second change rate threshold, the detection control unit 31 decreases the light emitting intensity of the light emitting element 11. As the light emitting intensity of the light emitting element 11 decreases, the variation in the AC component of the detection signal decreases, and the variation in the pulse wave decreases. As the variation in the pulse wave decreases, a decrease in the measurement accuracy of the pulse wave is prevented. In addition, a decrease in the measurement accuracy of the oxygen saturation concentration is prevented.

After the detection control unit 31 adjusts the light emitting intensity of the light emitting element 11, the data processing unit 33 acquires a detection signal based on the light emitted at the adjusted light emitting intensity. The data processing unit 33 measures a pulse wave and an oxygen saturation concentration using the detection signal. The data processing unit 33 outputs the measured pulse wave and oxygen saturation concentration to the display control unit 35, the communication interface 60, or the like.

The memory 40 stores various types of data. The memory 40 stores control data for operating various units, various types of data measured by the control unit 30, or the like. The memory 40 stores various evaluation values such as the change rate threshold and the correlation coefficient threshold. The memory 40 may store the pulse wave, the oxygen saturation concentration, or the like measured by the data processing unit 33. The memory 40 stores the control program CP that operates in the control unit 30. The memory 40 may store the correlation coefficient calculation formula. The memory 40 stores the calibration table PT to be referred to by the data processing unit 33. The memory 40 may store a conversion formula or a conversion table. The memory 40 includes a ROM, a RAM, or the like. The memory 40 corresponds to an example of a storage unit.

The control program CP is executed by the control unit 30 to operate various functional units. The control program CP causes the control unit 30 to operate as the detection control unit 31, the data processing unit 33, the display control unit 35, and the determination unit 37. The control program CP may operate the control unit 30 as functional units other than the detection control unit 31, the data processing unit 33, the display control unit 35, and the determination unit 37.

The calibration table PT is a table that stores the fluctuation component amplitude ratio and the oxygen saturation concentration in association with each other. The calibration table PT shows a relationship between the fluctuation component amplitude ratio and the oxygen saturation concentration. The calibration table PT is created in advance by a manufacturer of the measurement device 100. The data processing unit 33 determines the oxygen saturation concentration corresponding to the calculated fluctuation component amplitude ratio by referring to the calibration table PT.

The memory 40 may store a calibration formula instead of the calibration table PT. The calibration formula is a relational formula between the fluctuation component amplitude ratio and the oxygen saturation concentration. The data processing unit 33 measures the oxygen saturation concentration corresponding to the calculated fluctuation component amplitude ratio using the calibration formula.

The display panel 4 displays various images. The display panel 4 displays the pulse wave, the oxygen saturation concentration, or the like under the control of the display control unit 35. The display panel 4 may display the reliability data under the control of the display control unit 35. The display panel 4 displays a pulse rate based on the display data output from the display control unit 35. The display panel 4 may display the oxygen saturation concentration or the like. The display panel 4 includes a liquid crystal display, an organic electro-luminescence (EL) display, or the like.

The temperature detection sensor 50 measures the outside air temperature or the body temperature of the user M. The temperature detection sensor 50 provided in the first measurement device 100a measures the body temperature of the user M. The temperature detection sensor 50 measures the outside air temperature or the body temperature of the user M to generate temperature data. The temperature data is set data of temperatures measured at predetermined time intervals. The temperature detection sensor 50 may be of a contact type or a non-contact type. When the temperature detection sensor 50 is of a contact type, a thermocouple, a platinum temperature measuring resistor, or the like are provided in the detector 50S. The thermocouple, the platinum temperature measuring resistor, or the like are in contact with the user M. When the temperature detection sensor 50 is of a non-contact type, an irradiation unit that emits an infrared ray is provided in the detector 50S. The irradiation unit emits an infrared ray to the user M. The temperature detection sensor 50 outputs the temperature data to the control unit 30. The temperature detection sensor 50 corresponds to an example of a temperature sensor.

The communication interface 60 is an interface circuit that communicates with the tablet terminal 200. The communication interface 60 is connected to the tablet terminal 200 in a wired or wireless manner in accordance with a predetermined protocol. The communication interface 60 includes, for example, a connection port for wired communication, an antenna for wireless communication, or the like. The communication interface 60 receives control data, information related to the user M, or the like from the tablet terminal 200. The communication interface 60 transmits various types of biological information such as the pulse wave and the oxygen saturation concentration to the tablet terminal 200. The communication interface 60 transmits various types of measurement data such as the red light detection signal and the infrared light detection signal. The communication interface 60 may communicate with an external device other than the tablet terminal 200. The communication interface 60 corresponds to an example of a communication unit.

The battery 70 supplies power to various units or the like of the first measurement device 100a. The battery 70 includes a lithium primary battery, a lithium ion secondary battery, or the like. The battery 70 is preferably a rechargeable lithium ion secondary battery. The lithium ion secondary battery is charged in a wired or wireless manner.

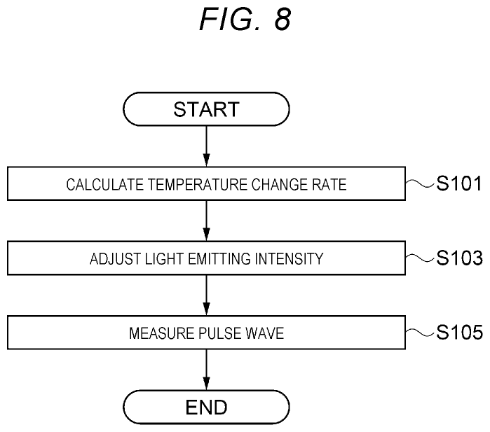

FIG. 8 shows a flowchart for measuring a pulse wave. The flowchart shown in FIG. 8 shows an example of a biological information measurement method. FIG. 8 shows a biological information measurement method executed by the first measurement device 100a. The first measurement device 100a may measure an oxygen saturation concentration.

In step S101, the control unit 30 calculates a temperature change rate. The temperature detection sensor 50 measures a body surface temperature of the user M and generates temperature data. The determination unit 37 of the control unit 30 acquires the temperature data from the temperature detection sensor 50. The determination unit 37 calculates the temperature change rate based on the temperature data. The determination unit 37 calculates a temperature change per unit time as the temperature change rate.

After calculating the temperature change rate, the control unit 30 adjusts the light emitting intensity of the light emitting element 11 in step S103. The determination unit 37 determines whether the temperature change rate is a positive value or a negative value. When the temperature change rate is a negative value, the determination unit 37 compares the temperature change rate with the first change rate threshold. When the temperature change rate is a positive value, the determination unit 37 compares the temperature change rate with the second change rate threshold. The determination unit 37 may compare the temperature change rate with the absolute value of the change rate threshold. The determination unit 37 causes the detection control unit 31 to adjust the light emitting intensity of the light emitting element 11 based on the determination result. The detection control unit 31 increases or decreases the light emitting intensity of the light emitting element 11 based on the temperature change rate. The detection control unit 31 adjusts at least one of the light emitting intensities of the red light emitting element 11a and the infrared light emitting element 11b. An adjustment amount of the light emitting intensity may be the same or different for both the red light emitting element 11a and the infrared light emitting element 11b. The adjustment amount of the light emitting intensity is preferably different between the red light emitting element 11a and the infrared light emitting element 11b. The adjustment amount of the light emitting intensity is stored in advance in the memory 40 in association with the temperature change rate.

After adjusting the light emitting intensity, the control unit 30 measures a pulse wave in step S105. The detection control unit 31 causes the light emitting element 11 to emit light with the adjusted light emitting intensity. The light emitting element 11 emits light with the adjusted light emitting intensity. The light receiving element unit 20 generates a detection signal. The data processing unit 33 acquires the detection signal. The data processing unit 33 measures the pulse wave based on the detection signal. The data processing unit 33 measures the pulse wave based on the red light detection signal or the infrared light detection signal. The data processing unit 33 may measure the oxygen saturation concentration based on the red light detection signal and the infrared light detection signal. The data processing unit 33 outputs the pulse wave and the oxygen saturation concentration to the display control unit 35 or the communication interface 60.

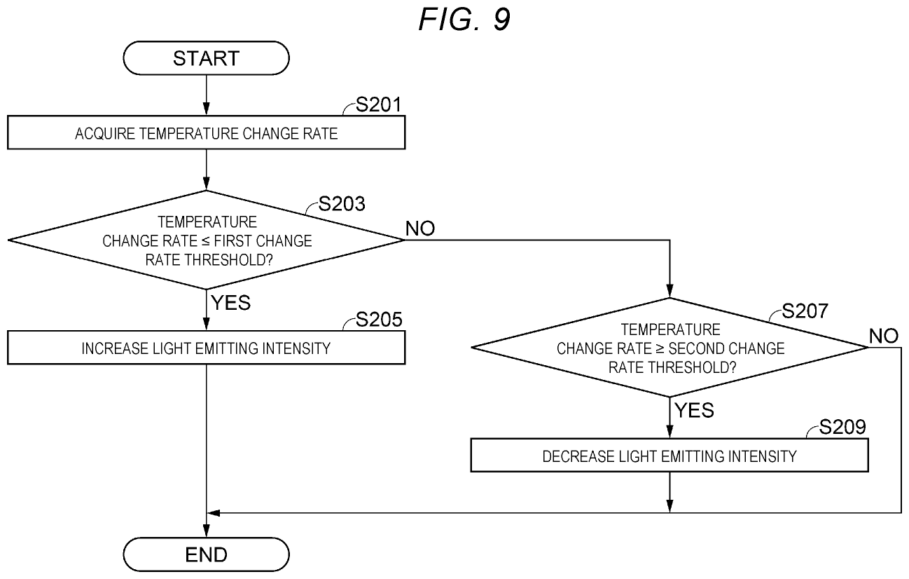

FIG. 9 shows a flowchart for adjusting a light emitting intensity. The flowchart shown in FIG. 9 shows details of a method of adjusting the light emitting intensity performed in step S103 of FIG. 8. FIG. 9 shows an example of the method of adjusting the light emitting intensity. FIG. 9 shows an adjustment method performed by the determination unit 37 and the detection control unit 31 in the control unit 30.

In step S201, the determination unit 37 of the control unit 30 acquires a temperature change rate. The determination unit 37 acquires the temperature change rate based on the temperature data acquired from the temperature detection sensor 50.

After acquiring the temperature change rate, the determination unit 37 compares the temperature change rate with the first change rate threshold in step S203. The first change rate threshold is a negative value. When the temperature change rate is a negative value and is equal to or smaller than the first change rate threshold, the determination unit 37 determines to increase the light emitting intensity of the light emitting element 11. The control unit 30 proceeds to step S205 (step S203: YES). When the temperature change rate is a positive value or greater than the first change rate threshold, the control unit 30 proceeds to step S207 (step S203: NO).

In step S205, the detection control unit 31 of the control unit 30 increases the light emitting intensity. The detection control unit 31 increases the light emitting intensity of the light emitting element 11. The detection control unit 31 increases the light emitting intensities of the red light emitting element 11a and the infrared light emitting element 11b. The adjustment amount of the light emitting intensity of the red light emitting element 11a is preferably different from the adjustment amount of the light emitting intensity of the infrared light emitting element 11b. The adjustment amount of the light emitting intensity of the red light emitting element 11a and the adjustment amount of the light emitting intensity of the infrared light emitting element 11b are predetermined corresponding to the temperature change rate.

In step S207, the determination unit 37 of the control unit 30 compares the temperature change rate with the second change rate threshold. The second change rate threshold is a positive value. When the temperature change rate is a positive value and is equal to or greater than the second change rate threshold, the determination unit 37 determines to decrease the light emitting intensity of the light emitting element 11. The control unit 30 proceeds to step S209 (step S207: YES). When the temperature change rate is smaller than the second change rate threshold, the determination unit 37 determines not to change the light emitting intensity of the light emitting element 11. The control unit 30 ends the adjustment of the light emitting intensity of the light emitting element 11 (step S207: NO).

In step S209, the detection control unit 31 of the control unit 30 decreases the light emitting intensity. The detection control unit 31 decreases the light emitting intensity of the light emitting element 11. The detection control unit 31 decreases the light emitting intensities of the red light emitting element 11a and the infrared light emitting element 11b. The adjustment amount of the light emitting intensity of the red light emitting element 11a is preferably different from the adjustment amount of the light emitting intensity of the infrared light emitting element 11b. The adjustment amount of the light emitting intensity of the red light emitting element 11a and the adjustment amount of the light emitting intensity of the infrared light emitting element 11b are predetermined corresponding to the temperature change rate.

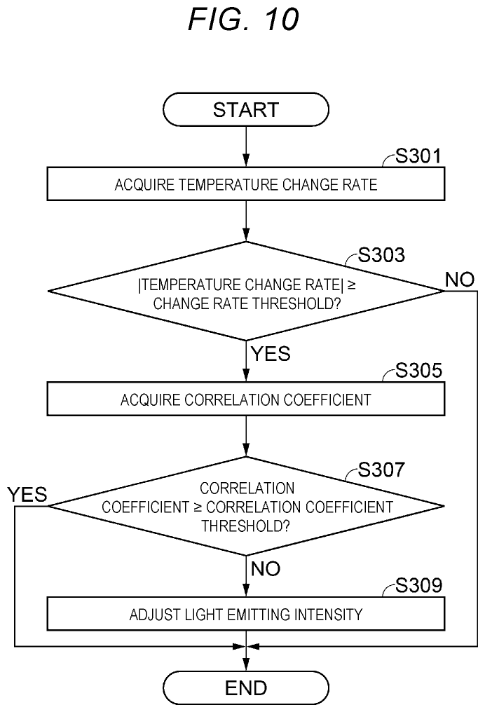

The control unit 30 may adjust the light emitting intensity of the light emitting element 11 based on the temperature change rate and the correlation coefficient. FIG. 10 shows a flowchart for adjusting the light emitting intensity. FIG. 10 shows a method of adjusting the light emitting intensity different from that of FIG. 9.

In step S301, the determination unit 37 of the control unit 30 acquires a temperature change rate. The determination unit 37 acquires the temperature change rate based on the temperature data acquired from the temperature detection sensor 50.

After acquiring the temperature change rate, the determination unit 37 compares the absolute value of the temperature change rate with the change rate threshold in step S303. When the absolute value of the temperature change rate is equal to or greater than the change rate threshold, the control unit 30 proceeds to step S305 (step S303: YES). When the absolute value of the temperature change rate is smaller than the change rate threshold, the control unit 30 ends the adjustment of the light emitting intensity of the light emitting element 11 (step S303: NO).

In step S305, the determination unit 37 acquires the correlation coefficient. The data processing unit 33 acquires the red light detection signal and the infrared light detection signal when the temperature detection sensor 50 measures the temperature data. The data processing unit 33 calculates the correlation coefficient using the red light detection signal and the infrared light detection signal. The determination unit 37 acquires the correlation coefficient calculated by the data processing unit 33.

After calculating the correlation coefficient, the determination unit 37 compares the correlation coefficient with the correlation coefficient threshold in step S307. The determination unit 37 reads the correlation coefficient threshold from the memory 40. The correlation coefficient threshold is stored in the memory 40 in advance. When the determination unit 37 determines that the correlation coefficient is equal to or greater than the correlation coefficient threshold, the control unit 30 ends the adjustment of the light emitting intensity of the light emitting element 11 (step S307: YES). The detection control unit 31 does not change the light emitting intensities of the red light emitting element 11a and the infrared light emitting element 11b. When the determination unit 37 determines that the correlation coefficient is smaller than the correlation coefficient threshold, the control unit 30 proceeds to step S309 (step S307: NO). When the correlation coefficient is smaller than the correlation coefficient threshold, the signal intensity of the AC component of the red light detection signal or the signal intensity of the AC component of the infrared light detection signal is smaller than a predetermined amount. When the signal intensity of the AC component decreases, detection accuracy of the pulse wave decreases.

In step S309, the detection control unit 31 adjusts the light emitting intensity. The detection control unit 31 increases or decreases the light emitting intensity of the light emitting element 11. When the temperature change rate is a negative value, the detection control unit 31 increases the light emitting intensity of the light emitting element 11. When the temperature change rate is a positive value, the detection control unit 31 decreases the light emitting intensity of the light emitting element 11. The detection control unit 31 adjusts at least one of the light emitting intensity of the red light emitting element 11a and the light emitting intensity of the infrared light emitting element 11b. The adjustment amount of the light emitting intensity of the red light emitting element 11a is preferably different from the adjustment amount of the light emitting intensity of the infrared light emitting element 11b. By adjusting the light emitting intensity for each of the red light emitting element 11a and the infrared light emitting element 11b, the measurement accuracy of the pulse wave and the oxygen saturation concentration is improved.

The first measurement device 100a includes the first light emitting element unit 10a including the red light emitting element 11a that emits the red light RL, the first light receiving element unit 20a that receives the red light RL passing through the user M to generate the red light detection signal, the temperature detection sensor 50 that measures the body temperature of the user M and generates the temperature data, and the control unit 30 that adjusts the light emitting intensity of the red light emitting element 11a. The control unit 30 calculates the temperature change rate by using the temperature data, and adjusts the light emitting intensity based on the temperature change rate.

The detection signal fluctuates depending on a change in body temperature. The first measurement device 100a can prevent a decrease in measurement accuracy of the pulse wave or the like by adjusting the light emitting intensity when the body temperature changes.

When the temperature change rate is a negative value, the control unit 30 preferably increases the light emitting intensity.

When the body temperature of the user M decreases, a detection intensity of the detection signal can be secured by increasing the light emitting intensity. A decrease in measurement accuracy of the pulse wave or the like is prevented.

When the temperature change rate is a positive value, the control unit 30 preferably decreases the light emitting intensity.

When the body temperature of the user M increases, signal variation in the detection signal can be prevented by decreasing the light emitting intensity. A decrease in measurement accuracy of the pulse wave or the like is prevented.

The first light emitting element unit 10a includes the infrared light emitting element 11b that emits the infrared light NL having a wavelength different from that of the red light RL. The first light receiving element unit 20a generates the infrared light detection signal by receiving the infrared light NL. The control unit 30 calculates the correlation coefficient between the red light detection signal and the infrared light detection signal, and adjusts the light emitting intensity based on the correlation coefficient.

By using the temperature change rate and the correlation coefficient, it is possible to adjust the light emitting intensity while checking the detection intensity of the detection signal.

The biological information measurement method is a biological information measurement method of measuring the biological information of the user M. The biological information measurement method includes: measuring the body temperature of the user M to generate the temperature data; emitting light to the user M; receiving light passing through the user M and generating the detection signal; calculating the temperature change rate using the temperature data; and adjusting, based on the temperature change rate, the light emitting intensity when light is emitted.

The detection signal fluctuates depending on a change in body temperature. By adjusting the light emitting intensity when the body temperature changes, the measurement accuracy of the pulse wave or the like can be prevented from decreasing.

### Second Embodiment

A second embodiment shows a configuration of a second measurement device 100b which is an example of the measurement device 100. The second embodiment shows a biological information measurement method using the second measurement device 100b. The second measurement device 100b according to the second embodiment measures a pulse wave and an oxygen saturation concentration. The second measurement device 100b transmits the measured pulse wave and oxygen saturation concentration to the tablet terminal 200. The tablet terminal 200 receives the pulse wave and the oxygen saturation concentration, and evaluates sleep apnea syndrome or the like.

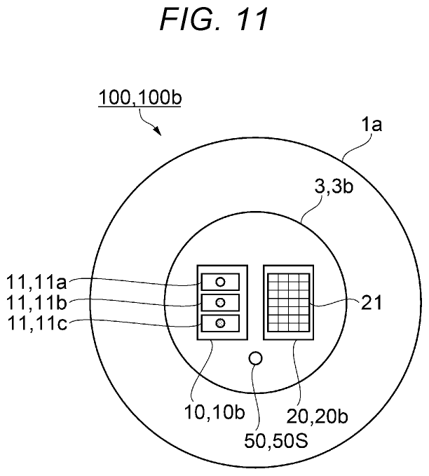

FIG. 11 shows a schematic configuration of the measurement surface 1a of the measurement device 100. FIG. 11 shows a schematic configuration when the measurement surface 1a of the second measurement device 100b, which is an example of the measurement device 100, is viewed from the outside. A second detection unit 3b is disposed on the measurement surface 1a. The second detection unit 3b is an example of the detection unit 3. The second detection unit 3b includes a second light emitting element unit 10b and a second light receiving element unit 20b. The second light emitting element unit 10b is an example of the light emitting element unit 10. The second light receiving element unit 20b is an example of the light receiving element unit 20. The second detection unit 3b includes the detector 50S of the temperature detection sensor 50.

The second light emitting element unit 10b emits light toward the measurement site of the user M. The second light emitting element unit 10b includes three light emitting elements 11. The three light emitting elements 11 are the red light emitting element 11a, the infrared light emitting element 11b, and a green light emitting element 11c. The green light emitting element 11c emits light in a wavelength range different from those of the red light emitting element 11a and the infrared light emitting element 11b.

The second light receiving element unit 20b receives various types of light emitted by the second light emitting element unit 10b. The second light receiving element unit 20b includes the light receiving element 21 that receives various types of light. The light receiving element 21 receives transmitted light or reflected light of the light emitted by the second light emitting element unit 10b.

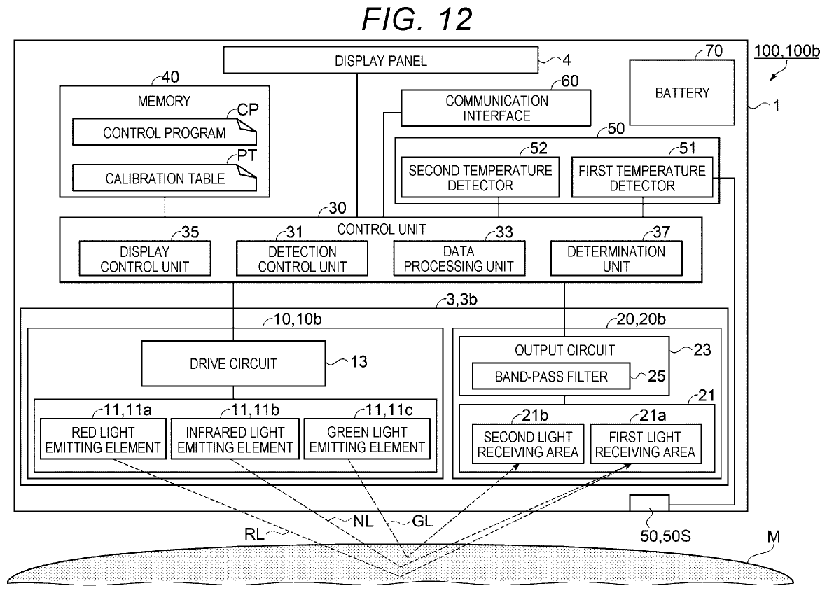

FIG. 12 shows a block configuration of the measurement device 100. FIG. 12 shows a block configuration of the second measurement device 100b which is an example of the measurement device 100. FIG. 12 shows the second measurement device 100b excluding the belt 2. The second measurement device 100b accommodates various units or the like in the housing 1. The second measurement device 100b includes the second detection unit 3b, the control unit 30, the memory 40, the display panel 4, the temperature detection sensor 50, the communication interface 60, and the battery 70. The control unit 30, the memory 40, the display panel 4, the communication interface 60, and the battery 70 of the second measurement device 100b are the same as the control unit 30, the memory 40, the display panel 4, the communication interface 60, and the battery 70 of the first measurement device 100a. The temperature detection sensor 50 of the second measurement device 100b includes a first temperature detector 51 and a second temperature detector 52.

The first temperature detector 51 measures the body temperature of the user M. The first temperature detector 51 measures the body surface temperature of the user M as the body temperature, and generates first temperature data. The first temperature data is an example of temperature data. The first temperature data is set data of the body surface temperatures of the user M measured at predetermined time intervals. The first temperature detector 51 is coupled to the detector 50S that is in contact with the user M. The detector 50S is provided at a position facing the user M. The first temperature detector 51 outputs the first temperature data to the control unit 30.

The second temperature detector 52 measures the outside air temperature of the second measurement device 100b. The outside air temperature corresponds to an example of an environmental temperature. The second temperature detector 52 measures the outside air temperature and generates second temperature data. The second temperature data is an example of temperature data. The second temperature data is set data of the outside air temperatures measured at predetermined time intervals. A measurement unit of the second temperature detector 52 is provided at a position not in contact with the user M. The measurement unit is provided in a portion of the housing 1. The measurement unit is not shown. The second temperature detector 52 outputs the second temperature data to the control unit 30.

The second measurement device 100b causes either the first temperature detector 51 or the second temperature detector 52 to measure the outside air temperature or the body temperature of the user M. The second measurement device 100b causes the first temperature detector 51 and the second temperature detector 52 to measure the outside air temperature and the body temperature of the user M. The second measurement device 100b uses at least one of the first temperature detector 51 and the second temperature detector 52 to generate at least one of the first temperature data and the second temperature data as the temperature data.

The drive circuit 13, the red light emitting element 11a, and the infrared light emitting element 11b of the second detection unit 3b are the same as the drive circuit 13, the red light emitting element 11a, and the infrared light emitting element 11b of the first detection unit 3a. The second detection unit 3b includes the second light emitting element unit 10b including the green light emitting element 11c.

The green light emitting element 11c emits green light GL in a wavelength range from 520 nm to 550 nm toward the measurement site of the user M. The green light GL is, for example, light having a peak wavelength of 520 nm. The green light emitting element 11c corresponds to an example of a third light emitting element. The green light emitting element 11c is driven by the drive circuit 13 similarly to the red light emitting element 11a and the infrared light emitting element 11b.

The second light receiving element unit 20b provided in the second detection unit 3b includes the light receiving element 21 and the output circuit 23. The light receiving element 21 receives light emitted by the light emitting element 11 and reflected by the measurement site of the user M. The light receiving element 21 receives the red light RL, the infrared light NL, and the green light GL reflected by the measurement site of the user M. The light receiving element 21 is divided into a plurality of regions. The light receiving element 21 may be divided into a plurality of regions using an optical filter (not shown). The light receiving element 21 shown in FIG. 12 is divided into a first light receiving area 21a and a second light receiving area 21b.

The first light receiving area 21a receives the red light RL and the infrared light NL. The first light receiving area 21a receives the red light RL emitted by the red light emitting element 11a and reflected by the measurement site of the user M. The first light receiving area 21a receives the infrared light NL emitted by the infrared light emitting element 11b and reflected by the measurement site of the user M. The first light receiving area 21a may receive at least one of the red light RL and the infrared light NL via the optical filter. The first light receiving area 21a may alternately receive the red light RL and the infrared light NL in a time-division manner.

The second light receiving area 21b receives the green light GL. The second light receiving area 21b receives the green light GL emitted by the green light emitting element 11c and reflected by the measurement site of the user M. The second light receiving area 21b may receive the green light GL via the optical filter.

In FIG. 12, the red light RL and the infrared light NL are received by the first light receiving area 21a, but are not limited thereto. A third light receiving area different from the first light receiving area 21a and the second light receiving area 21b may be provided. The red light RL or the infrared light NL may be received by the third light receiving area. When the third light receiving area receives the infrared light NL, the first light receiving area 21a receives the red light RL. The light receiving element 21 may not be divided into the plurality of regions. The light receiving element 21 may receive the red light RL, the infrared light NL, and the green light GL in a time-division manner.

The output circuit 23 outputs, to the control unit 30, a detection signal based on the light received by the light receiving element 21. The output circuit 23 generates a detection signal by performing processing such as analog-to-digital conversion on light receiving intensity data of the light received by the light receiving element 21. The output circuit 23 generates a red light detection signal based on the red light RL received by the first light receiving area 21a. The output circuit 23 generates an infrared light detection signal based on the infrared light NL received by the first light receiving area 21a. The output circuit 23 generates a green light detection signal based on the green light GL received by the second light receiving area 21b. The green light detection signal is an example of the detection signal.

The output circuit 23 may include a band-pass filter 25. The band-pass filter 25 extracts an AC component from the light receiving intensity data. The band-pass filter 25 separates the light receiving intensity data into an AC component and a DC component by extracting the AC component from the light receiving intensity data. The AC component corresponds to the AC component data 83 shown in FIG. 5. The DC component corresponds to the DC component data 81 shown in FIG. 5. The band-pass filter 25 outputs the separated AC component and DC component as detection signals to the control unit 30.

The band-pass filter 25 extracts a red light AC component from the red light RL received by the first light receiving area 21a. The band-pass filter 25 separates the light receiving intensity data into the red light AC component and a red light DC component by extracting the red light AC component. The band-pass filter 25 extracts an infrared light AC component from the infrared light NL received by the first light receiving area 21a. The band-pass filter 25 separates the light receiving intensity data into the infrared light AC component and an infrared light DC component by extracting the infrared light AC component. The output circuit 23 outputs the red light AC component and the red light DC component as red light detection signals to the control unit 30. The output circuit 23 outputs the infrared light AC component and the infrared light DC component as infrared light detection signals to the control unit 30.

The band-pass filter 25 extracts a green light AC component from the green light GL received by the second light receiving area 21b. The band-pass filter 25 separates the light receiving intensity data into the green light AC component and a green light DC component by extracting the green light AC component. The output circuit 23 outputs the green light AC component and the green light DC component as green light detection signals to the control unit 30.

Similarly to the first measurement device 100a, the control unit 30 of the second measurement device 100b measures an oxygen saturation concentration using the red light detection signal and the infrared light detection signal. The control unit 30 of the second measurement device 100b performs the same control as the control unit 30 of the first measurement device 100a to measure the oxygen saturation concentration.

The control unit 30 of the second measurement device 100b measures a pulse wave using the green light detection signal. The green light detection signal is less likely to be affected by the body movement or the like of the user M than the red light detection signal and the infrared light detection signal. The second measurement device 100b can improve the measurement accuracy of the pulse wave by measuring the pulse wave using the green light detection signal.

The determination unit 37 acquires at least one of the first temperature data and the second temperature data. The determination unit 37 acquires the first temperature data from the first temperature detector 51. Alternatively, the determination unit 37 acquires the second temperature data from the second temperature detector 52. The determination unit 37 may acquire the first temperature data from the first temperature detector 51 and acquire the second temperature data from the second temperature detector 52. The determination unit 37 calculates a temperature change rate by using at least one of the first temperature data and the second temperature data.

The determination unit 37 may calculate a detection signal ratio of the green light detection signal. The determination unit 37 adjusts the light emitting intensity of the green light emitting element 11c based on the temperature change rate and the detection signal ratio of the green light detection signal. For example, when the absolute value of the temperature change rate is equal to or greater than the change rate threshold, the determination unit 37 calculates the detection signal ratio of the green light detection signal. The determination unit 37 compares the detection signal ratio with a predetermined ratio threshold. When the detection signal ratio is smaller than the ratio threshold, the determination unit 37 causes the detection control unit 31 to adjust a light emitting intensity of the green light emitting element 11c. The detection signal ratio corresponds to an example of the signal ratio. The ratio threshold corresponds to an example of the predetermined value.

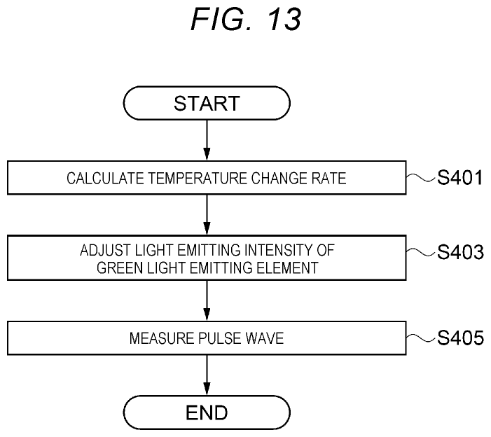

FIG. 13 shows a flowchart for measuring a pulse wave. The flowchart shown in FIG. 13 shows an example of a biological information measurement method. FIG. 13 shows a biological information measurement method executed by the second measurement device 100b. The second measurement device 100b may measure an oxygen saturation concentration using the red light detection signal and the infrared light detection signal.

In step S401, the control unit 30 calculates a temperature change rate. The first temperature detector 51 measures the body surface temperature of the user M and generates the first temperature data. The second temperature detector 52 may measure the outside air temperature and generate the second temperature data. The determination unit 37 of the control unit 30 acquires at least one of the first temperature data and the second temperature data. The determination unit 37 calculates the temperature change rate based on at least one of the first temperature data and the second temperature data. The determination unit 37 calculates a temperature change per unit time as the temperature change rate.

After calculating the temperature change rate, the control unit 30 adjusts the light emitting intensity of the green light emitting element 11c in step S403. The determination unit 37 determines whether the temperature change rate is a positive value or a negative value. When the temperature change rate is a negative value, the determination unit 37 compares the temperature change rate with the first change rate threshold. When the temperature change rate is a positive value, the determination unit 37 compares the temperature change rate with the second change rate threshold. The determination unit 37 may compare the temperature change rate with the absolute value of the change rate threshold. The determination unit 37 causes the detection control unit 31 to adjust the light emitting intensity of the green light emitting element 11c based on the determination result. The detection control unit 31 increases or decreases the light emitting intensity of the green light emitting element 11c based on the temperature change rate. The detection control unit 31 may adjust at least one of the light emitting intensities of the red light emitting element 11a and the infrared light emitting element 11b.

After adjusting the light emitting intensity of the green light emitting element 11c, the control unit 30 measures the pulse wave in step S405. The detection control unit 31 causes the green light emitting element 11c to emit light with the adjusted light emitting intensity. The green light emitting element 11c emits light with the adjusted light emitting intensity. The second light receiving element unit 20b generates the green light detection signal. The data processing unit 33 acquires the green light detection signal. The data processing unit 33 measures the pulse wave based on the green light detection signal. The detection control unit 31 may cause the red light emitting element 11a and the infrared light emitting element 11b to emit light with the adjusted light emitting intensity. The red light emitting element 11a and the infrared light emitting element 11b emit light with the adjusted light emitting intensity. The second light receiving element unit 20b generates the red light detection signal and the infrared light detection signal. The data processing unit 33 measures the oxygen saturation concentration based on the red light detection signal and the infrared light detection signal. The data processing unit 33 outputs the pulse wave and the oxygen saturation concentration to the display control unit 35 or the communication interface 60.

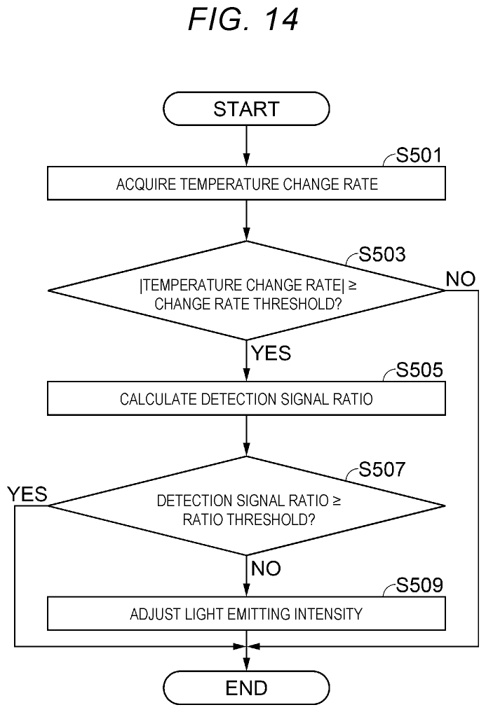

The control unit 30 may adjust the light emitting intensity of the green light emitting element 11c based on the temperature change rate and the detection signal ratio of the green light detection signal. FIG. 14 shows a flowchart for adjusting the light emitting intensity. The control unit 30 may adjust the light emitting intensity of the red light emitting element 11a and the light emitting intensity of the infrared light emitting element 11b based on the temperature change rate and the detection signal ratio. The control unit 30 calculates the detection signal ratio of the red light detection signal and the detection signal ratio of the infrared light detection signal. The control unit 30 adjusts the light emitting intensity of the red light emitting element 11a based on the temperature change rate and the detection signal ratio of the red light detection signal. The control unit 30 adjusts the light emitting intensity of the infrared light emitting element 11b based on the temperature change rate and the detection signal ratio of the infrared light detection signal.

In step S501, the determination unit 37 of the control unit 30 acquires a temperature change rate. The determination unit 37 acquires the temperature change rate based on at least one of the first temperature data and the second temperature data.

After acquiring the temperature change rate, the determination unit 37 compares the absolute value of the temperature change rate with the change rate threshold in step S503. When the absolute value of the temperature change rate is equal to or greater than the change rate threshold, the control unit 30 proceeds to step S505 (step S503: YES). When the absolute value of the temperature change rate is smaller than the change rate threshold, the control unit 30 ends the adjustment of the light emitting intensity of the green light emitting element 11c (step S503: NO).

In step S505, the determination unit 37 acquires the detection signal ratio. The determination unit 37 acquires the green light detection signal when the temperature detection sensor 50 measures the temperature data. The determination unit 37 calculates the detection signal ratio using a DC component and an AC component of the green light detection signal. The determination unit 37 may calculate the detection signal ratio of the red light detection signal and the detection signal ratio of the infrared light detection signal.

After calculating the detection signal ratio, the determination unit 37 compares the detection signal ratio with the ratio threshold in step S507. The determination unit 37 reads the ratio threshold from the memory 40. The ratio threshold is stored in advance in the memory 40. When the determination unit 37 determines that the detection signal ratio of the green light detection signal is equal to or greater than the ratio threshold, the control unit 30 ends the adjustment of the light emitting intensity of the green light emitting element 11c (step S507: YES). The detection control unit 31 does not change the light emitting intensity of the green light emitting element 11c. When the determination unit 37 determines that the detection signal ratio of the green light detection signal is smaller than the ratio threshold, the control unit 30 proceeds to step S509 (step S507: NO). When the detection signal ratio is smaller than the ratio threshold, the signal intensity of the AC component of the detection signal is smaller than the predetermined amount. When the signal intensity of the AC component decreases, the measurement accuracy of the pulse wave decreases. The determination unit 37 may compare the ratio threshold with each of the detection signal ratio of the red light detection signal and the detection signal ratio of the infrared light detection signal.

In step S509, the detection control unit 31 adjusts the light emitting intensity. The detection control unit 31 increases or decreases the light emitting intensity of the green light emitting element 11c. When the temperature change rate is a negative value, the detection control unit 31 increases the light emitting intensity of the green light emitting element 11c. When the temperature change rate is a positive value, the detection control unit 31 decreases the light emitting intensity of the green light emitting element 11c. The detection control unit 31 may adjust the light emitting intensity of the red light emitting element 11a and the light emitting intensity of the infrared light emitting element 11b.

The second measurement device 100b includes the second light emitting element unit 10b including the green light emitting element 11c that emits the green light GL, the second light receiving element unit 20b that receives the green light GL passing through the user M to generate the green light detection signal, the temperature detection sensor 50 that measures at least one of the outside air temperature and the body temperature of the user M and generates at least one of the first temperature data and the second temperature data as the temperature data, and the control unit 30 that adjusts the light emitting intensity of the green light emitting element 11c. The control unit 30 calculates the temperature change rate by using at least one of the first temperature data and the second temperature data, and adjusts the light emitting intensity based on the temperature change rate.

The green light detection signal fluctuates depending on a change in body temperature. The second measurement device 100b can prevent a decrease in measurement accuracy of the pulse wave or the like by adjusting the light emitting intensity when the body temperature changes.

The control unit 30 calculates a detection signal ratio of the AC component to the DC component of the green light detection signal, and adjusts the light emitting intensity based on the calculated detection signal ratio.

By using the temperature change rate and the detection signal ratio, the second measurement device 100b can adjust the light emitting intensity while checking a detection intensity of the green light detection signal.

When the detection signal ratio is smaller than the ratio threshold, the control unit 30 preferably increases the light emitting intensity.

When the detection signal ratio is smaller than the ratio threshold, the AC component of the green light detection signal is smaller than the predetermined amount. The second measurement device 100b can improve the detection accuracy of the pulse wave or the like by increasing the light emitting intensity.

### Third Embodiment

A third embodiment shows an example of a measurement system 1000 that analyzes an irregular pulse or the like in the tablet terminal 200. The measurement system 1000 according to the third embodiment includes the second measurement device 100b and the tablet terminal 200. The measurement system 1000 may include the first measurement device 100a instead of the second measurement device 100b.

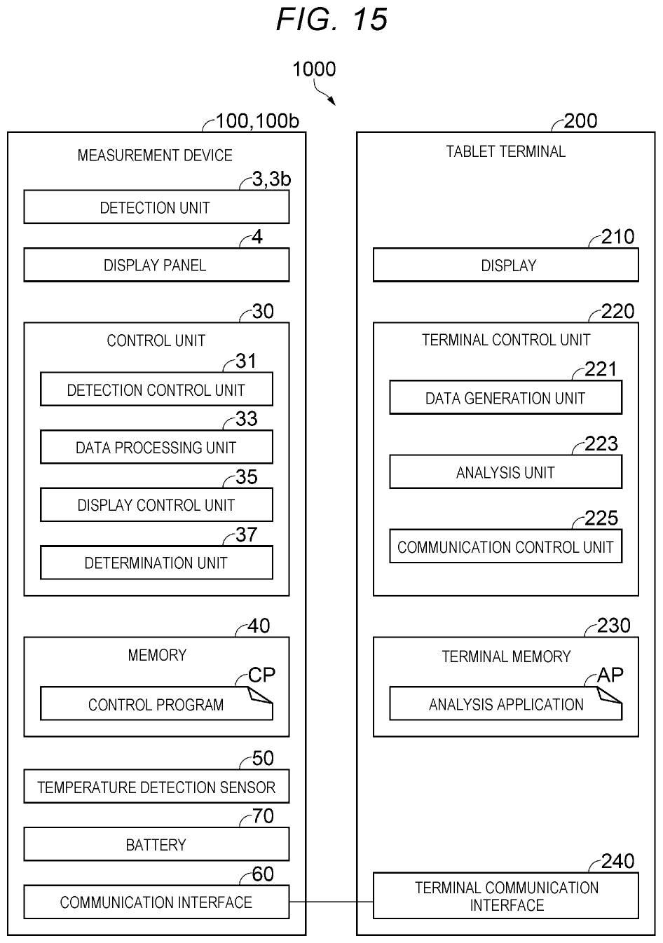

FIG. 15 shows a block configuration of the measurement system 1000. A configuration of the second measurement device 100b shown in FIG. 15 is the same as the configuration of the second measurement device 100b shown in FIG. 12. The second measurement device 100b generates a detection signal under the control of the control unit 30. The control unit 30 generates a green light detection signal or the like. The second measurement device 100b transmits the green light detection signal to the tablet terminal 200 via the communication interface 60. The communication interface 60 transmits the green light detection signal to the tablet terminal 200. The control unit 30 may generate a red light detection signal and an infrared light detection signal. The second measurement device 100b transmits the red light detection signal and the infrared light detection signal to the tablet terminal 200 via the communication interface 60. The communication interface 60 transmits the red light detection signal and the infrared light detection signal to the tablet terminal 200.

The tablet terminal 200 can calculate a pulse wave and an oxygen saturation concentration. The tablet terminal 200 evaluates the irregular pulse based on the pulse wave. The tablet terminal 200 evaluates sleep apnea syndrome based on the oxygen saturation concentration. The tablet terminal 200 includes the display 210, the terminal control unit 220, a terminal memory 230, and a terminal communication interface 240.

The terminal control unit 220 is a terminal controller that controls operations of various units in the tablet terminal 200. The terminal control unit 220 analyzes biological information of the user M. The terminal control unit 220 is, for example, a terminal processor including a CPU. The terminal control unit 220 may include one or a plurality of processors. The terminal control unit 220 may include a semi-conductor memory such as a RAM or a ROM. The semi-conductor memory functions as a work area of the terminal control unit 220. The terminal control unit 220 functions as a data generation unit 221, an analysis unit 223, and a communication control unit 225 by executing an analysis application AP stored in the terminal memory 230.

The data generation unit 221 is a functional unit that operates in the terminal control unit 220. The data generation unit 221 calculates biological information such as the pulse wave and the oxygen saturation concentration. When the green light detection signal is acquired from the second measurement device 100b, the data generation unit 221 calculates the pulse wave using the green light detection signal. When the red light detection signal or the infrared light detection signal is acquired from the second measurement device 100b, the data generation unit 221 may calculate the pulse wave using the red light detection signal or the infrared light detection signal. When the red light detection signal and the infrared light detection signal are acquired from the second measurement device 100b, the data generation unit 221 calculates the oxygen saturation concentration using the red light detection signal and the infrared light detection signal. The data generation unit 221 calculates the oxygen saturation concentration by the same method as the method executed by the data processing unit 33. The data generation unit 221 outputs the calculated pulse wave and oxygen saturation concentration to the analysis unit 223.

The analysis unit 223 is a functional unit that operates in the terminal control unit 220. The analysis unit 223 acquires the pulse wave output from the data generation unit 221. The analysis unit 223 evaluates the irregular pulse by analyzing the pulse wave. The analysis unit 223 outputs an evaluation result of the irregular pulse to the display 210. The analysis unit 223 may cause the terminal memory 230 to store the evaluation result of the irregular pulse.

The analysis unit 223 acquires the biological information such as the oxygen saturation concentration output from the data generation unit 221. For example, the analysis unit 223 analyzes the oxygen saturation concentration to evaluate the sleep apnea syndrome. The analysis unit 223 outputs an evaluation result of the sleep apnea syndrome to the display 210. The analysis unit 223 may cause the terminal memory 230 to store the evaluation result of the sleep apnea syndrome. The analysis unit 223 may generate chart data using the oxygen saturation concentration. The analysis unit 223 outputs the generated chart data to the display 210. The display 210 displays various charts based on the chart data.

The communication control unit 225 is a functional unit that operates in the terminal control unit 220. The communication control unit 225 controls communication with the second measurement device 100b. The communication control unit 225 establishes communication connection with the second measurement device 100b. The communication control unit 225 causes the second measurement device 100b to transmit various types of data at a predetermined timing. The communication control unit 225 causes the second measurement device 100b to transmit the red light detection signal, the infrared light detection signal, and the green light detection signal at predetermined timings.

The terminal memory 230 stores various types of data. The terminal memory 230 stores control data for operating various units in the tablet terminal 200. The terminal memory 230 may store various types of analysis data or the like analyzed by the terminal control unit 220. The terminal memory 230 stores the analysis application AP operating in the terminal control unit 220. The terminal memory 230 stores the calibration table PT referred to by the data generation unit 221. The terminal memory 230 includes a ROM, a RAM, or the like. The terminal memory 230 corresponds to an example of a terminal storage unit.

The analysis application AP is executed by the terminal control unit 220 to operate various functional units. The analysis application AP causes the terminal control unit 220 to operate as the data generation unit 221, the analysis unit 223, and the communication control unit 225. The analysis application AP may cause the terminal control unit 220 to operate as functional units other than the data generation unit 221, the analysis unit 223, and the communication control unit 225.

The terminal communication interface 240 is a terminal interface circuit that communicates with the second measurement device 100b. The terminal communication interface 240 is coupled to the second measurement device 100b in a wired or wireless manner in accordance with a predetermined protocol. The terminal communication interface 240 includes, for example, a connection port for wired communication, an antenna for wireless communication, or the like. The terminal communication interface 240 receives various types of data such as the red light detection signal, the infrared light detection signal, and the green light detection signal from the second measurement device 100b. The terminal communication interface 240 transmits, to the second measurement device 100b, various types of control data for controlling the operation of the second measurement device 100b, information related to the user M, or the like. The terminal communication interface 240 may communicate with an external device other than the second measurement device 100b. The terminal communication interface 240 corresponds to an example of a terminal communication circuit.

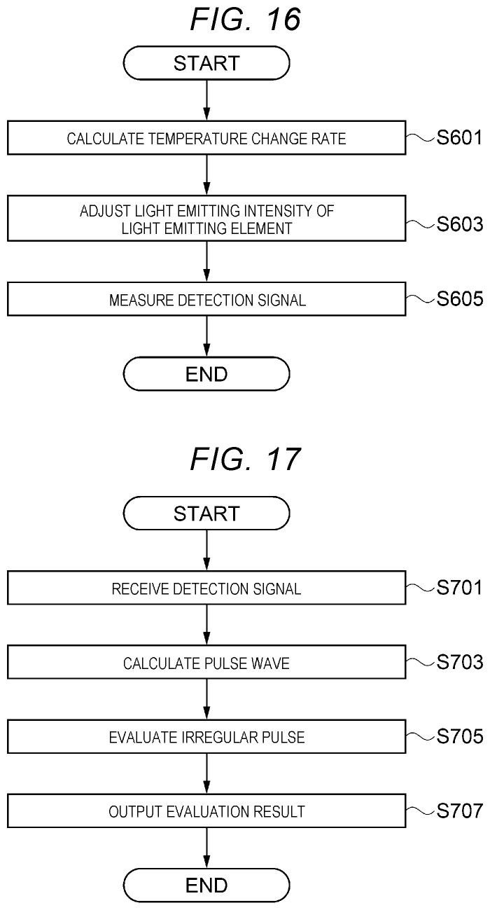

FIG. 16 is a flowchart for measuring a detection signal. The flowchart shown in FIG. 16 shows a part of the biological information measurement method. FIG. 16 shows a method of measuring the detection signal executed by the second measurement device 100b. The second measurement device 100b adjusts the light emitting intensity of the green light emitting element 11c to generate the green light detection signal. The second measurement device 100b may adjust the light emitting intensity of the red light emitting element 11a and the light emitting intensity of the infrared light emitting element 11b to measure the red light detection signal and the infrared light detection signal.

In step S601, the control unit 30 calculates a temperature change rate. The first temperature detector 51 measures the body surface temperature of the user M and generates the first temperature data. The second temperature detector 52 may measure the outside air temperature and generate the second temperature data. The determination unit 37 of the control unit 30 acquires at least one of the first temperature data and the second temperature data. The determination unit 37 calculates the temperature change rate based on at least one of the first temperature data and the second temperature data.

After calculating the temperature change rate, the control unit 30 adjusts the light emitting intensity of the light emitting element 11 in step S603. The detection control unit 31 of the control unit 30 adjusts the light emitting intensity of the green light emitting element 11c. The determination unit 37 determines whether the temperature change rate is a positive value or a negative value. When the temperature change rate is a negative value, the determination unit 37 compares the temperature change rate with the first change rate threshold. When the temperature change rate is a positive value, the determination unit 37 compares the temperature change rate with the second change rate threshold. The determination unit 37 may compare the temperature change rate with the absolute value of the change rate threshold. The determination unit 37 causes the detection control unit 31 to adjust the light emitting intensity of the green light emitting element 11c based on the determination result. The detection control unit 31 increases or decreases the light emitting intensity of the green light emitting element 11c based on the temperature change rate. The detection control unit 31 may adjust at least one of the light emitting intensities of the red light emitting element 11a and the infrared light emitting element 11b.

After adjusting the light emitting intensity of the light emitting element 11, the control unit 30 measures the detection signal in step S605. The detection control unit 31 causes the green light emitting element 11c to emit light with the adjusted light emitting intensity. The green light emitting element 11c emits light with the adjusted light emitting intensity. The second light receiving element unit 20b generates the green light detection signal. The data processing unit 33 acquires the green light detection signal. The detection control unit 31 may cause the red light emitting element 11a and the infrared light emitting element 11b to emit light with the adjusted light emitting intensity. The red light emitting element 11a and the infrared light emitting element 11b emit light with the adjusted light emitting intensity. The second light receiving element unit 20b generates the red light detection signal and the infrared light detection signal. The data processing unit 33 acquires the red light detection signal and the infrared light detection signal. The data processing unit 33 outputs the green light detection signal to the tablet terminal 200 via the communication interface 60. The data processing unit 33 outputs the red light detection signal and the infrared light detection signal to the tablet terminal 200 via the communication interface 60.

FIG. 17 shows an operation of the tablet terminal 200 in the measurement system 1000. FIG. 17 is a flowchart showing the operation of the tablet terminal 200 according to the third embodiment. The flowchart shown in FIG. 17 shows a part of the biological information measurement method executed by the measurement system 1000.

In step S701, the tablet terminal 200 receives a detection signal. The terminal communication interface 240 of the tablet terminal 200 receives the detection signal transmitted via the communication interface 60. The terminal communication interface 240 receives the green light detection signal as the detection signal. The terminal communication interface 240 receives the red light detection signal and the infrared light detection signal. The terminal communication interface 240 receives the green light detection signal or the like at a predetermined timing. The terminal communication interface 240 transmits the red light detection signal, the infrared light detection signal, and the green light detection signal to the data generation unit 221.

After receiving the detection signal, the tablet terminal 200 calculates a pulse wave in step S703. The data generation unit 221 receives the green light detection signal and calculates the pulse wave using the green light detection signal. The data generation unit 221 receives the red light detection signal and the infrared light detection signal, and calculates an oxygen saturation concentration using the red light detection signal and the infrared light detection signal. The oxygen saturation concentration is calculated by the data generation unit 221 in the same method as the method executed by the data processing unit 33 according to the first embodiment. The data generation unit 221 transmits the generated pulse wave and oxygen saturation concentration to the analysis unit 223.

After calculating the pulse wave, the tablet terminal 200 evaluates an irregular pulse in step S705. The analysis unit 223 analyzes the pulse wave. The analysis unit 223 evaluates the irregular pulse by analyzing the pulse wave. The analysis unit 223 transmits an evaluation result of the irregular pulse as an analysis result to the display 210 or the terminal memory 230.

In step S705, the tablet terminal 200 analyzes the biological information such as the oxygen saturation concentration. For example, the analysis unit 223 evaluates sleep apnea syndrome using the oxygen saturation concentration. The analysis unit 223 transmits an evaluation result of the sleep apnea syndrome to the display 210 or the terminal memory 230.

After evaluating the irregular pulse, the tablet terminal 200 outputs the analysis result in step S707. The display 210 displays the evaluation result of the irregular pulse as the output of the analysis result. The tablet terminal 200 may output the evaluation result of the irregular pulse by voice or the like. The tablet terminal 200 may display the analysis result and a chart indicating a change in pulse wave on the display 210.

In step S707, the tablet terminal 200 may output an analysis result of the biological information. For example, the tablet terminal 200 displays the evaluation result of the sleep apnea syndrome on the display 210. The tablet terminal 200 outputs evaluation results of various types of biological information calculated using the red light detection signal, the infrared light detection signal, and the green light detection signal.

The measurement system 1000 includes the second measurement device 100b and the tablet terminal 200. The second measurement device 100b includes the second light emitting element unit 10b including the green light emitting element 11c that emits the green light GL, the second light receiving element unit 20b that receives the green light GL passing through the user M to generate the green light detection signal, the temperature detection sensor 50 that measures at least one of the outside air temperature and the body temperature of the user M and generates at least one of the first temperature data and the second temperature data as the temperature data, the control unit 30 that calculates the temperature change rate by using at least one of the first temperature data and the second temperature data, and adjusts the light emitting intensity of the green light emitting element 11c based on the calculated temperature change rate, and the communication interface 60 that transmits the green light detection signal. The tablet terminal 200 includes the terminal communication interface 240 that receives the green light detection signal, and the terminal control unit 220 that analyzes biological information of the user M using the green light detection signal.

The green light detection signal fluctuates depending on a change in body temperature. The second measurement device 100b can prevent a decrease in measurement accuracy of the pulse wave or the like by adjusting the light emitting intensity when the body temperature changes. The measurement system 1000 can evaluate the irregular pulse or the like with high accuracy.

What is claimed is:

1. A biological information measurement device, comprising:
a light emitting unit including a light emitting element that emits light;
a light receiving unit configured to receive the light passing through a living body and generate a detection signal;
a temperature sensor configured to measure at least one of an environmental temperature and a body temperature of the living body and generate temperature data; and
a controller configured to adjust a light emitting intensity of the light emitting element, wherein
the controller is configured to
calculate a temperature change rate using the temperature data, and
adjust the light emitting intensity based on the temperature change rate.

2. The biological information measurement device according to claim 1, wherein
when the temperature change rate is a negative value, the controller increases the light emitting intensity.

3. The biological information measurement device according to claim 1, wherein
when the temperature change rate is a positive value, the controller decreases the light emitting intensity.

4. The biological information measurement device according to claim 1, wherein
the controller calculates a signal ratio of an AC component to a DC component of the detection signal, and adjusts the light emitting intensity based on the calculated signal ratio.

5. The biological information measurement device according to claim 4, wherein
when the signal ratio is smaller than a predetermined value, the controller increases the light emitting intensity.

6. The biological information measurement device according to claim 1, wherein
the light emitting unit includes a second light emitting element that emits second light having a wavelength different from that of the light,
the light receiving unit receives the second light and generates a second detection signal, and
the controller calculates a correlation coefficient between the detection signal and the second detection signal, and adjusts the light emitting intensity based on the correlation coefficient.

7. A biological information measurement method of measuring biological information of a living body, the biological information measurement method comprising:
measuring at least one of an environmental temperature and a body temperature of the living body and generating temperature data;
emitting light to the living body;
receiving the light passing through the living body and generating a detection signal;
calculating a temperature change rate using the temperature data; and
adjusting, based on the temperature change rate, a light emitting intensity when the light is emitted.

8. A biological information measurement system, comprising:
a biological information measurement device including:
a light emitting unit including a light emitting element that emits light,
a light receiving unit configured to receive the light passing through a living body and generate a detection signal,
a temperature sensor configured to measure at least one of an environmental temperature and a body temperature of the living body and generate temperature data,
a controller configured to calculate a temperature change rate using the temperature data, and adjust a light emitting intensity of the light emitting element based on the calculated temperature change rate, and
a communication unit configured to transmit the detection signal; and
a control device including:
a terminal communication circuit configured to receive the detection signal, and
an analysis controller configured to analyze biological information of the living body using the detection signal.
